(19) United States
(12) Patent Application Publication
(10) Pub. No.: US 2002/0049315 A1
(43) Pub. Date: Apr. 25, 2002

(54) Crystalline forms of carbapenem intermediates

(76) Inventor: John M. Williams, Belle Mead, NJ (US)

Correspondence Address: MERCK AND CO INC, P O BOX 2000, RAHWAY, NJ, 070650907

(21) Appl. No.: 09/921,221

(22) Filed: Aug. 2, 2001

Related U.S. Application Data
(60) Provisional application No. 60/230,605, filed on Sep. 6, 2000.

(51) Int. Cl. C07D 205/08

(52) U.S. Cl. 540/354

(57) ABSTRACT

Crystalline forms of [2R-[2α(R*),3β(R*)]]-3-(1-hydroxyethyl)-γ-methyl-β,4-dioxo-2-azetidinebutanoic acid(4-nitrophenyl)methyl ester and crystalline form of (γR,2R,3S)-α-diazo-3-[(1R)-1-hydroxyethyl]-γ-methyl-β,4-dioxo-2-azetidinebutanoic acid (4-nitrophenyl)methyl ester are disclosed.

### BACKGROUND OF THE INVENTION

Azetidinones are inherently unstable compounds that tend to hydrolyze. For this reason it is particularly advantageous to produce a stable crystalline form of any azetidinone that will be prepared on production scale requiring handling, storage, and shipping. Additionally, crystalline forms of intermediates for carbapenem antibiotics are desirable from a stability and purity standpoint. These compounds facilitate the synthesis of carbapenem antibiotics on a commercial scale.

In the present invention, crystalline forms of the compound [2R-[2α(R*),3β(R*)]]-3-(1-hydroxyethyl)-γ-methyl-β,4-dioxo-2-azetidinebutanoic acid (4-nitrophenyl)methyl and (γR,2R,3S)-α-diazo-3-[(1R)-1-hydroxyethyl]-β-methyl-,β,4-dioxo-2-azetidinebutanoic acid (4-nitrophenyl)methyl ester have been discovered and characterized. The compounds can generally be synthesized taking into account the disclosures of U.S. Pat. Nos. 4,312,871 granted on Jan. 26, 1982; 5,998,612, granted on Dec. 7, 1999; 4,350,631, granted on Sep. 21, 1982; 4,454,332, granted on Jun. 12, 1984; and 4,994,568, granted on Feb. 19, 1991, all herein incorporated by reference.

### SUMMARY OF THE INVENTION

Crystalline forms of [2R-[2α(R*),3β(R*)]]-3-(1-hydroxyethyl)-γ-methyl-β,4-dioxo-2-azetidinebutanoic acid (4-nitrophenyl)methyl ester (compound of formula I) and crystalline (γR,2R,3S)-α-diazo-3-[(1R)-1-hydroxyethyl]-γ-methyl-β,4-dioxo-2-azetidinebutanoic acid (4-nitrophenyl)methyl ester (compound of formula II) are disclosed.

### DESCRIPTION OF THE FIGURES

FIG. 1. depicts the X-ray powder diffraction (XRPD) pattern of Form A and Form B, the crystal forms of [2R-[2α(R*),3β(R*)]]-3-(1-hydroxyethyl)-γ-methyl-β,4-dioxo-2-azetidinebutanoic acid (4-nitrophenyl)methyl ester (formula I).

FIG. 2. Depicts the X-ray powder diffraction pattern of the crystal form of the compound of formula II, (γR,2R,3S)-α-diazo-3-[(1R)-1-hydroxyethyl]-γ-methyl-β,4-dioxo-2-azetidinebutanoic acid (4-nitrophenyl)methyl ester.

### DETAILED DESCRIPTION OF THE INVENTION

The compounds have the following structural formulas:

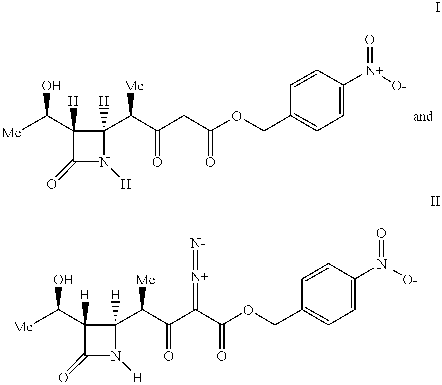

The crystalline forms of the compounds are characterized below by virtue of their X-Ray Powder Diffraction (XRPD) patterns. The XRPD patterns were collected using CuKα radiation with an accelerating potential of 45 kV and a filament emission of 40 mA. Diffraction patterns were collected from 2 to 40 °2Theta.

The crystalline Form A of the compound of formula I is unambiguously characterized as having an XRPD pattern at 7.9, 7.5, 7.0, 5.2, 4.8, 4.4, 3.9, 3.7, 3.6, 3.5, 3.3, 3.2, 3.1, 3.0, and 2.9 angstroms. More complete XRPD data pertaining to the compound is shown below in Table 1.

TABLE 1

| Angle | D Spacing | I/I max |
|---|---|---|
| (° 2 Theta) | (angstroms) | (%) |
| 11.2 | 7.87 | 35 |
| 11.7 | 7.53 | 21 |
| 11.9 | 7.46 | 18 |
| 12.8 | 6.92 | 22 |
| 14.0 | 6.32 | 7 |
| 15.4 | 5.74 | 8 |
| 17.1 | 5.19 | 62 |
| 17.7 | 5.00 | 8 |
| 18.6 | 4.75 | 53 |
| 19.7 | 4.51 | 38 |
| 20.4 | 4.35 | 100 |
| 20.9 | 4.24 | 10 |
| 22.2 | 4.00 | 32 |
| 23.0 | 3.86 | 69 |
| 23.9 | 3.72 | 43 |
| 25.0 | 3.55 | 28 |
| 25.5 | 3.49 | 38 |
| 26.2 | 3.39 | 6 |
| 27.2 | 3.28 | 24 |
| 27.7 | 3.22 | 29 |
| 29.0 | 3.08 | 25 |
| 29.4 | 3.03 | 38 |
| 30.7 | 2.91 | 21 |
| 31.6 | 2.83 | 8 |
| 32.7 | 2.74 | 7 |
| 33.3 | 2.68 | 9 |
| 34.0 | 2.64 | 10 |
| 34.6 | 2.59 | 6 |
| 35.9 | 2.50 | 8 |
| 36.5 | 2.46 | 10 |
| 37.5 | 2.40 | 19 |
| 38.3 | 2.35 | 17 |

Form A: The melting point was measured using DSC thermography. The DSC was determined using a heating rate of 10° C./min under a nitrogen atmosphere from room temperature to 200° C. A major endotherm (melting endotherm) was detected with a peak temperature of about 105° C., an extrapolated onset temperature of 103° C., and a heat of melting of 102 Joules/g.

The crystalline Form B of the compound of formula I is unambiguously characterized as having an XRPD pattern at 9.5, 7.8, 6.5, 5.9, 5.5, 4.9, 4.5, 4.3, 3.9, 3.8, 3.6, 3.5, 3.3, 3.1, 2.9 angstroms. More complete XRPD data pertaining to the compound is shown below in Table 2.

TABLE 2

| Angle | D Spacing | I/I max |
|---|---|---|
| (° 2 Theta) | (angstroms) | (%) |
| 9.4 | 9.45 | 24 |
| 11.3 | 7.83 | 15 |
| 12.6 | 7.01 | 11 |
| 13.2 | 6.68 | 38 |
| 13.6 | 6.52 | 81 |
| 15.0 | 5.90 | 81 |
| 16.0 | 5.55 | 94 |
| 16.1 | 5.50 | 42 |
| 16.9 | 5.23 | 10 |
| 18.1 | 4.90 | 57 |
| 18.5 | 4.78 | 30 |
| 19.6 | 4.51 | 65 |
| 20.5 | 4.34 | 43 |
| 22.1 | 4.02 | 49 |
| 22.8 | 3.90 | 58 |
| 23.6 | 3.76 | 33 |
| 24.3 | 3.65 | 37 |
| 25.4 | 3.50 | 100 |
| 26.9 | 3.31 | 41 |
| 28.5 | 3.13 | 54 |
| 28.8 | 3.10 | 35 |
| 30.9 | 2.89 | 72 |
| 31.5 | 2.83 | 21 |
| 32.5 | 2.76 | 29 |
| 33.5 | 2.68 | 25 |
| 34.4 | 2.60 | 32 |
| 35.1 | 2.56 | 25 |
| 37.0 | 2.43 | 20 |
| 38.3 | 2.35 | 20 |
| 39.4 | 2.28 | 37 |

Form B: The melting point was measured using DSC thermography. The DSC was determined using a heating rate of 10° C./min under a nitrogen atmosphere from room temperature to 200° C. A major endotherm (melting endotherm) was detected with a peak temperature of about 101° C., an extrapolated onset temperature of 99° C., and a heat of melting of 82 Joules/g.

The crystalline form of the compound of formula II is unambiguously characterized as having an XRPD pattern at 8.3, 5.7, 5.6, 5.4, 4.9, 4.5, 4.1, 4.0, 3.8, 3.7, 3.6, 3.5, 3.4, 3.2, 3.1, 3.0, 2.7, and 2.5 angstroms. More complete XRPD data pertaining to the solvate is shown below in Table 3.

TABLE 3

| Angle | D Spac | I/I max |
|---|---|---|
| (° 2 Theta) | (angstroms) | (%) |
| 10.7 | 8.26 | 27 |
| 11.1 | 8.00 | 5 |
| 11.7 | 7.58 | 9 |
| 12.8 | 6.91 | 6 |
| 14.0 | 6.30 | 15 |
| 14.5 | 6.11 | 6 |
| 15.5 | 5.71 | 70 |
| 15.9 | 5.58 | 67 |
| 16.2 | 5.47 | 28 |
| 16.6 | 5.35 | 83 |
| 18.1 | 4.91 | 84 |
| 18.3 | 4.85 | 39 |
| 19.0 | 4.68 | 11 |
| 19.8 | 4.49 | 54 |
| 21.4 | 4.14 | 62 |
| 22.4 | 3.97 | 30 |
| 23.5 | 3.79 | 61 |
| 23.9 | 3.72 | 68 |
| 24.5 | 3.63 | 100 |
| 25.3 | 3.52 | 44 |
| 25.8 | 3.45 | 60 |
| 26.5 | 3.36 | 59 |
| 26.8 | 3.33 | 28 |
| 27.2 | 3.27 | 20 |
| 28.0 | 3.19 | 31 |
| 28.4 | 3.14 | 39 |
| 29.3 | 3.05 | 30 |
| 30.6 | 2.92 | 9 |
| 31.9 | 2.80 | 19 |
| 32.6 | 2.75 | 24 |
| 33.8 | 2.65 | 9 |
| 34.6 | 2.59 | 16 |
| 35.6 | 2.52 | 22 |
| 36.2 | 2.48 | 19 |
| 36.6 | 2.45 | 12 |
| 37.1 | 2.42 | 6 |
| 37.8 | 2.38 | 9 |
| 38.3 | 2.34 | 9 |

The melting point was measured using DSC thermography. The DSC was determined using a heating rate of 10° C./min under a nitrogen atmosphere from room temperature to 140° C. A major endotherm (melting endotherm) was detected with a peak temperature of about 106° C., an extrapolated onset temperature of 102° C., and a heat of melting of 70 Joules/g.

The crystalline compounds of this invention are useful as intermediates in the preparation of carbapenem antibiotics, which are useful for the treatment of bacterial infections in animal and human subjects.

The compounds can be produced in accordance with the following non-limiting examples.

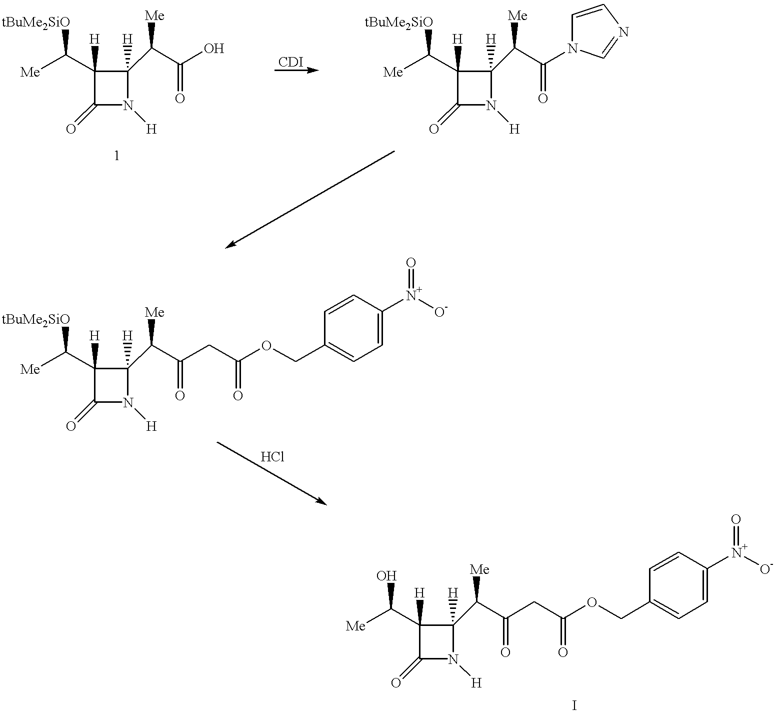

To a solution of the azetidinone carboxylic acid (1, prepared according to the teachings of Heterocycles 1984, 21, 29; U.S. Pat. Nos. 4,454,332 and 5,998,612 or a combination thereof) in acetonitrile (80 mL) was charged CDI (11.4 g) at 20° C. The resulting solution was added to a mixture containing acetonitrile (120 mL), magnesium chloride (5.7 g), and p-nitrobenzyl malonate (27.0 g), and the mixture was warmed to 45° C. After the reaction was complete, the mixture was cooled to 20° C. and 2.1 N HCl (114 mL) was added with agitation. The mixture was allowed to settle and the layers were separated. To the organic layer was added aqueous HCl (2.1 N, 22.8 mL) with agitation. Isopropyl acetate (230 mL) followed by 20% potassium bicarbonate (200 mL) were added and the layers were separated. The organic solution was washed with aqueous sodium chloride (5%, 100 mL) and water (50 mL), then concentrated to 100 mL under vacuum. Heptane (100 mL) was added to crystallize the product, which was isolated by filtration. Vacuum drying provided 19.4 g of product as a crystalline solid.

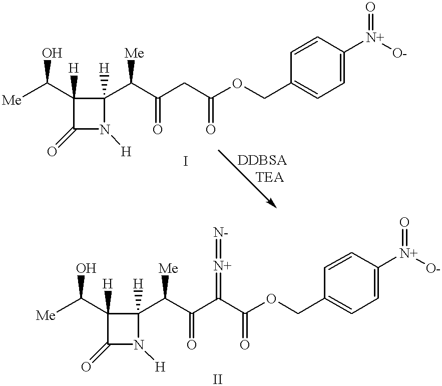

The compound of formula I (10.0 g) was dissolved in toluene (82 mL) and the resulting solution was added to a solution of dodecylbenzenesulfonylazide (DDBSA) in toluene (295 g/L, 38 mL). Triethylamine (0.5 mL) was added while maintain a temperature of below 25° C. The mixture was aged then seeded. The resulting slurry was aged then heptane (240 mL) was added to crystallize the product, which was isolated by filtration. Drying under vacuum afforded 8.6 g of product.

What is claimed is:

1. Crystalline [2R-[2α(R*),3β(R*)]]-3-(1-hydroxyethyl)-γ-methyl-β,4-dioxo-2-azetidinebutanoic acid (4-nitrophenyl)methyl ester, which is characterized by a solid state x-ray powder diffraction (XRPD) pattern having the following d-spacings: 7.9, 7.5, 7.0, 5.2, 4.8, 4.4, 3.9, 3.7, 3.6, 3.5, 3.3, 3.2, 3.1, 3.0, and 2.9 angstroms.

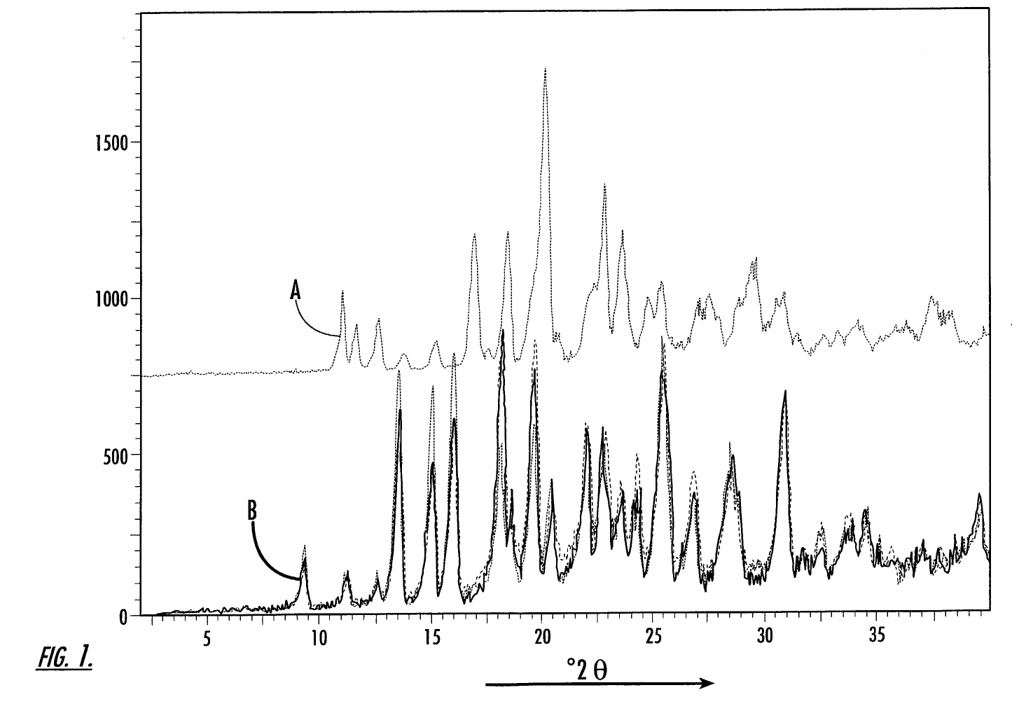

2. A crystal according to claim 1, having an x-ray powder diffraction pattern in accordance with FIG. 1, Form A.

3. A crystal according to claim 1, which is further characterized by an endotherm with an extrapolated onset temperature of 103° C., a peak temperature of about 105° C., and a heat of melting of 102 Joules/g.

4. Crystalline [2R-[2α(R*),3β(R*)]]-3-(1-hydroxyethyl)-γ-methyl-β,4-dioxo-2-azetidinebutanoic acid (4-nitrophenyl)methyl ester, which is characterized by a solid state x-ray powder diffraction (XRPD) pattern having the following d-spacings: 9.5, 7.8, 6.5, 5.9, 5.5, 4.9, 4.5, 4.3, 3.9, 3.8, 3.6, 3.5, 3.3, 3.1, 2.9 angstroms.

5. A crystal according to claim 4, having an x-ray powder diffraction pattern in accordance with FIG. 1, Form B.

6. A crystal according to claim 4, which is further characterized by an endotherm with an extrapolated onset temperature of 99° C., a peak temperature of about 101° C., and a heat of melting of 82 Joules/g.

7. Crystalline (γR,2R,3S)-α-diazo-3-[(1R)-1-hydroxyethyl]-γ-methyl-β,4-dioxo-2-azetidinebutanoic acid (4-nitrophenyl)methyl ester, which is characterized by a solid state x-ray powder diffraction (XRPD) pattern having the following d-spacings: 8.3, 5.7, 5.6, 5.4, 4.9, 4.5, 4.1, 4.0, 3.8, 3.7, 3.6, 3.5, 3.4, 3.2, 3.1, 3.0, 2.7, and 2.5 angstroms.

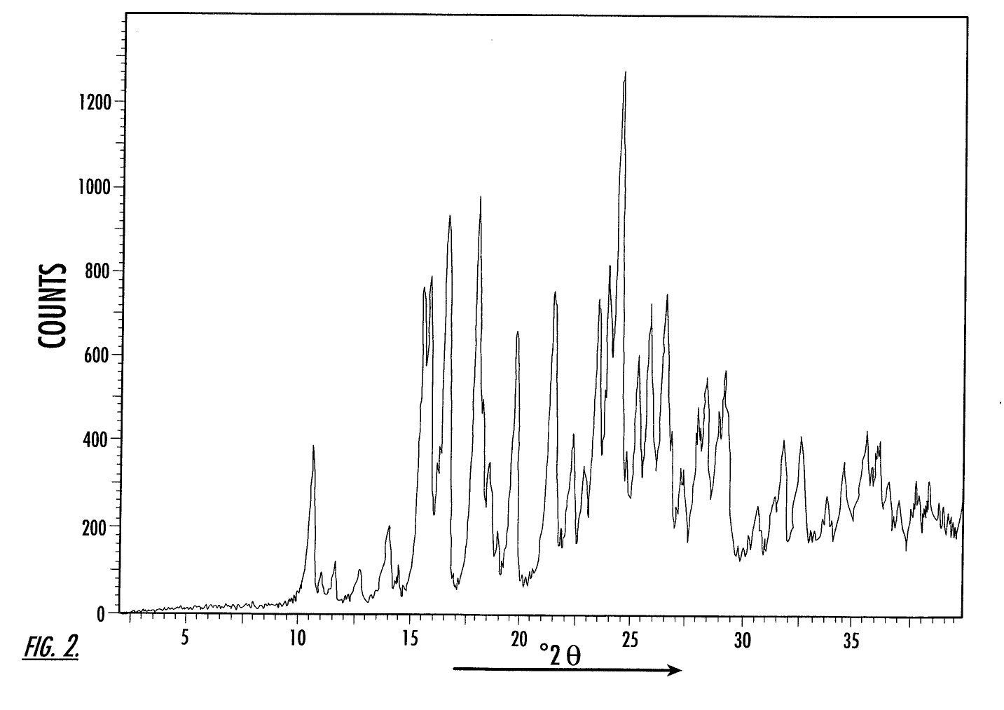

8. A crystal according to claim 7, having an x-ray powder diffraction pattern in accordance with FIG. 2.

9. A crystal according to claim 7, which is further characterized by an endotherm with an extrapolated onset temperature of 102° C., a peak temperature of about 106° C., and a heat of melting of 70 Joules/g.
